SEQUENCE LISTING (2 sequences)

SEQ ID NO: 1 - DNA, 250 bp, Gluconobacter cerinus
cgaagggggctagcgttgctcggaatgactgggcgtaaagggcgcgtaggcggtttatgcagtcagatgtgaaatccccgggcttaacctgggaactgcatttgagacgcatagactagaggtcgagagagggttgtggaattcccagtgtagaggtgaaattcgtagatattgggaagaacaccggtggcgaaggcggcaacctggctcgatactgacgctgaggcgcgaaagcgtggggagcaaacag

SEQ ID NO: 2 - DNA, 364 bp, Hanseniaspora uvarum
agtcgtaacaaggtttccgtaggtgaacctgcggaaggatcattagattgaattatcattgttgctcgagttcttgtttagatcttttacaataatgtgtatctttattgaagatgtgcgcttaattgcgctgcttctttaaagtgtcgcagtgaaagtagtcttgcttgaatctcagtcaacgctacacacattggagtttttttactttaatttaattctttctgctttgaatcgaaaggttcaaggcaaaaaacaaacacaaacaattttattttattataattttttaaactaaaccaaaattcctaacggaaattttaaaataatttaaaactttcaacaacggatctcttggttctct

(19) United States
(12) Patent Application Publication
(10) Pub. No.: US 2022/0256861 A1
(43) Pub. Date: Aug. 18, 2022

(54) MICROBIAL COMPOSITIONS FOR THE PREVENTION OR REDUCTION OF GROWTH OF FUNGAL PATHOGENS ON PLANTS

(71) Applicant: Boost Biomes, Inc., Brisbane, CA (US)

(72) Inventors: Veronica Garcia, Brisbane, CA (US); Sophia Andrikopoulos, Brisbane, CA (US); Jensina Froland, Brisbane, CA (US); Kelly Trinidad, Brisbane, CA (US); Christy Piamonte, Brisbane, CA (US); James Pearce, Brisbane, CA (US); Jamie Bacher, Brisbane, CA (US); Nathaniel T. Becker, Palo Alto, CA (US); Aleksandra Virag, Palo Alto, CA (US); Amruta J. Bedekar, Palo Alto, CA (US); Elizabeth A. Malinich, Palo Alto, CA (US)

(21) Appl. No.: 17/669,888

(22) Filed: Feb. 11, 2022

Related U.S. Application Data
(63) Continuation of application No. PCT/US2020/046165, filed on Aug. 13, 2020.
(60) Provisional application No. 62/886,883, filed on Aug. 14, 2019.

(51) Int. Cl. A01N 63/20; C12N 1/20; A23B 7/155

(52) U.S. Cl. CPC A01N 63/20; A23B 7/155; C12N 1/20

(57) ABSTRACT

Disclosed herein are biocontrol compositions against plant fungal pathogens and methods of use thereof for the prevention or reduction of crop loss or food spoilage.

### CROSS-REFERENCE

This application is a continuation Application of International Application No. PCT/US2020/046165, filed Aug. 13, 2020, which claims priority to U.S. Provisional Application No. 62/886,883, filed Aug. 14, 2019, each of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Feb. 6, 2022, is named 51401-704.301_SL.txt and is 1,418 bytes in size.

### BACKGROUND

Fungal pathogens cause significant agricultural loss, leading to loss of crops, food waste and economic loss. Microbes having anti-fungal properties have been developed as biological control agents to reduce both crop loss and food spoilage by these fungal pathogens. Commercially available products may not show the desired plant or fungal specificity or effectiveness. Furthermore, there are limited options for post-harvest protection of produce, particularly organic produce. Biocontrol compositions to prevent fungal growth can provide alternatives to currently available products.

### SUMMARY

Provided herein are biocontrol compositions for preventing or reducing fungal pathogen growth or infection in plants, and methods of making and using the same.

In an aspect the present disclosure provides a biocontrol composition comprising at least two microbes, wherein the at least two microbes comprise a Gluconobacter cerinus; and a Hanseniaspora uvarum, wherein the at least two microbes are co-cultured, wherein the at least two microbes are co-cultured at a product ratio. In some embodiments, the product ratio of the Gluconobacter cerinus and the Hanseniaspora uvarum is between about 1:100 and 100:1. In some embodiments, the product ratio of the Gluconobacter cerinus and the Hanseniaspora uvarum is between about 1:10 and 10:1. In some embodiments, the product ratio of the Gluconobacter cerinus and the Hanseniaspora uvarum is between about 1:5 and 5:1. In some embodiments, the product ratio of the Gluconobacter cerinus and the Hanseniaspora uvarum is between about 1:3 and 3:1. In some embodiments, the product ratio of the Gluconobacter cerinus and the Hanseniaspora uvarum is between about 1:2 and 2:1.

In some embodiments, the biocontrol composition is capable of inhibiting a fungal disease incidence by 10% or more compared to a reference composition comprising any composition selected from the group consisting of: (i) one or more of the at least two microbes cultured individually or (ii) the at least two microbes cultured separately and combined at a viable cell count and product ratio that is about the same as that of the biocontrol composition. In some embodiments, a viable cell count at the end of fermentation of the co-cultured at least two microbes, grown using a given fermentation medium, feed composition and process, is more than five times the sum of the viable cell counts of the at least two microbes grown alone in the equivalent fermentation process. In some embodiments, a viable cell count at the end of fermentation of the co-cultured at least two microbes, grown using a given fermentation medium, feed composition and process, is more than three times than a sum of the viable cell counts of the at least two microbes at the end of an equivalent fermentation process. In some embodiments, a viable cell count at the end of fermentation of the co-cultured at least two microbes, grown using a given fermentation medium, feed composition and process, is more than two times than a sum of the viable cell counts of the at least two microbes at the end of an equivalent fermentation process. In some embodiments, a viable cell count of the at least two microbes after being subjected to a storage condition, is higher than a sum of viable cell counts of the at least two microbes grown alone in an equivalent fermentation process and under the storage condition. In some embodiments, wherein the storage condition comprises storage at a temperature between 4° C. and 25° C. In some embodiments, the storage condition comprises a storage time of at least 7 days.

In another aspect, the present disclosure provides a method for generating a biocontrol composition, wherein the method comprises: (a) introducing a first microbe of the at least two microbes to a first culturing medium; (b) introducing a second microbe of the at least two microbes to a second culturing medium, wherein the second culturing medium comprises: the first culturing medium or a derivative thereof, the first microbe, or a combination thereof, wherein the second microbe is different from the first microbe; and (c) subjecting the first microbe and second microbe to conditions to allow cell proliferation, thereby generating the biocontrol composition. In some embodiments, the second culturing medium is the first culturing medium after conditioning by the first microbe. In some embodiments, the first microbe is Gluconobacter cerinus and the second microbe is Hanseniaspora uvarum. In some embodiments, the first microbe is Hanseniaspora uvarum and the second microbe is Gluconobacter cerinus.

In another aspect, the present disclosure provides a method of reducing or preventing growth of a pathogen on a plant, a seed, a flower or produce thereof comprising: applying any of the biocontrol compositions to the plant, seed, flower or produce thereof. In some embodiments, the plant, seed, flower, or produce thereof is selected from the group consisting of alfafa, almond, apricot, apple, artichoke, banana, barley, beet, blackberry, blueberry, broccoli, Brussels sprout, cabbage, cannabis, canola, capsicum, carrot, celery, chard, cherry, citrus, corn, cotton, cucurbit, date, fig, flax, garlic, grape, herb, spice, kale, lettuce, mint, oil palm, olive, onion, pea, pear, peach, peanut, papaya, parsnip, pecan, persimmon, plum, pomegranate, potato, quince, radish, raspberry, rose, rice, sloe, sorghum, soybean, spinach, strawberry, sweet potato, tobacco, tomato, turnip greens, walnut, and wheat. In some embodiments, the plant, seed, flower, or produce thereof comprises a strawberry.

In another aspect, the present disclosure provides a method of reducing or preventing the growth of a pathogen on a produce comprising: applying a biocontrol composition to a packaging material used to transport or store a produce. In some embodiments, the produce is selected from the group consisting of alfafa, almond, apricot, apple, artichoke, banana, barley, beet, blackberry, blueberry, broccoli, Brussels sprout, cabbage, cannabis, canola, capsicum, carrot, celery, chard, cherry, citrus, corn, cotton, cucurbit, date, fig, flax, garlic, grape, herb, spice, kale, lettuce, mint, oil palm, olive, onion, pea, pear, peach, peanut, papaya, parsnip, pecan, persimmon, plum, pomegranate, potato, quince, radish, raspberry, rose, rice, sloe, sorghum, soybean, spinach, strawberry, sweet potato, tobacco, tomato, turnip greens, walnut, and wheat. In some embodiments, the produce is a strawberry.

In another aspect, the present disclosure provides a method of reducing or preventing the growth of a pathogen on a strawberry fruit comprising applying a biocontrol compositions to a packaging material used to transport or store the strawberry fruit.

In various aspects, the pathogen is selected from the group consisting of: Albugo candida, Albugo occidentalis, Alternaria alternata, Alternaria cucumerina, Alternaria dauci, Alternaria solani Alternaria tenuis, Alternaria tenuissima, Alternaria tomatophila, Aphanomyces euteiches, Aphanomyces raphani, Armillaria mellea, Aspergillus flavus, Aspergillus parasiticus, Botrydia theobromae, Botrytis cinerea, Botrytinia fuckeliana, Bremia lactuca, Cercospora beticola, Cercosporella rubi, Cladosporium herbarum, Colletotrichum acutatum, Colletotrichum gloeosporioides, Colletotrichum lindemuthianum, Colletotrichum musae, Colletotrichum spaethanium, Cordana musae, Corynespora cassiicola, Daktuflosphaira Didymella bryoniae, Elsinoe ampelina, Elsinoe mangiferae, Elsinoe veneta, Erysiphe cichoracearum, Erysiphe necator, Eutypa lata, Fusarium germinareum, Fusarium oxysporum, Fusarium solani, Fusarium virguliforme, Gaeumannomyces graminis, Ganoderma boninense, Geotrichum candidum, Guignardia bidwellii, Gymnoconia peckiana, Helminthosporium solani, Leptosphaeria coniothyrium, Leptosphaeria maculans, Leveillula taurica, Macrophomina phaseolina, Microsphaera alni, Monilinia fructicola, Monilinia vaccinii-corymbosi, Mycosphaerella angulate, Mycosphaerella brassicicola, Mycosphaerella fragariae, Mycosphaerella fijiensis, Oidopsis taurica, Passalora fulva, Peronospora sparse, Peronospora farinosa, Pestalotiopsis clavispora, Phoma exigua, Phomopsis obscurans, Phomopsis vaccinia, Phomopsis viticola, Phytophthora capsica, Phytophthora erythroseptica, Phytophthora infestans, Phytophthora parasitica, Phytophthora ramorum, Plasmopara viticola, Plasmodiophora brassicae, Podosphaera macularis, Polyscytalum pustulans, Pseudocercospora vitis, Puccinia allii, Puccinia sorghi, Pucciniastrum vaccinia, Pythium aphanidermatum, Pythium debaryanum, Pythium sulcatum, Pythium ultimum, Ralstonia solanacearum, Ramularia tulasneii, Rhizoctonia solani, Rhizopus arrhizus, Rhizopus stoloniferz, Sclerotinia minor, Sclerotinia homeocarpa, Sclerotium cepivorum, Sclerotium rolfsii, Sclerotinia minor, Sclerotinia sclerotiorum, Septoria apiicola, Septoria lactucae, Septoria lycopersici, Septoria petroelini, Sphaceloma perseae, Sphaerotheca macularis, Spongospora subterrannea, Stemphylium vesicarium, Synchytrium endobioticum, Thielaviopsis basicola, Uncinula necator, Uromyces appendiculatus, Uromyces betae, Verticillium albo-atrum, Verticillium dahliae, Verticillium theobromae, and any combination thereof. In some embodiments, the pathogen is Botrytis cinerea.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings of which:

FIG. 1 illustrates BC18 inhibition of Botrytis, as measured by ‘LBDI’ (Local Botrytis Disease Incidence) on strawberry fruits. A low LBDI represents inhibition of Botrytis by the treatment. BC18B and BC18Y refer to the isolated bacterial and yeast components of BC18, respectively. Sterilized strawberries are treated before the experiment, while Non-sterilized strawberries include the baseline infection of Botryis. ‘C’ and ‘R’ illustrate Co-fermented and Recombined, respectively, and 1:1 and 3:1 are ratios of bacteria: yeast components of BC18.

FIGS. 2A-2F shows BC18 LBDI on strawberries. FIG. 2A shows the efficacy of 3:1 co-cultured BC18. FIG. 2B shows the efficacy of combined 3:1 BC18. FIG. 2C shows the efficacy of 1:1 co-cultured BC18. FIG. 2D shows the efficacy of combined 1:1 BC18. FIG. 2E shows the efficacy of yeast cultured individually. FIG. 2F shows reference images for LBDI of strawberries receiving no BC18 inoculation.

FIGS. 3A-3F show a visual representation of a Health Score scale used to quantify fungal disease incidence (FDI). A high FDI indicates protective effects of the treatment. FIG. 3A shows 4-point strawberry fruit which has no fungal disease evident. FIG. 3B shows a 3-point strawberry fruit. FIG. 3C shows a 2-point strawberry. FIG. 3D shows a 1-point strawberry. FIG. 3E shows another 1-point strawberry. FIG. 3F shows a 0-point strawberry.

FIG. 4 shows BC18 efficacy against fungal disease incidence (FDI) on strawberries.

FIG. 5 illustrates a flow cytometry distribution analysis of microbial cell populations.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Numerous fungal pathogens can infect plants of agricultural importance, resulting in food rot and food spoilage while the plants are in the field or after being harvested. For example, Grey Mold, caused by the fungal pathogen Botrytis cinerea, can often be found on fruits, such as strawberries and raspberries, both in the field and at the grocery store. Finding ways to reduce loss caused by fungal pathogens is highly desirable by anyone involved in food production and consumption, and chemical- and biological-based control strategies have previously been developed. However, the use of chemical- and biological-based fungicides on food crops, while effective, can provide unintended side effects (e.g., toxicity) in addition to being undesirable from a consumer standpoint.

In particular, there is a need for biocontrol composition with superior anti-fungal efficacy, and high viable cell count at the end of culturing and in liquid or dry formulations after extended storage at ambient or refrigerated conditions.

Disclosed herein are compositions and methods of use thereof, which compositions comprise at least one microbe (i.e. microbial strain) and a carrier. In many cases, there may be no single microbial strain that, by itself, provides adequate effective control of fungal pathogens on crops, on the plant, on fruit or other plant parts, during field cultivation, or for post-harvest protection of produce. In many cases, a single microbial strain may exhibit evidence of strong control of fungal pathogens in laboratory cultures, such as in confronting a culture of fungal pathogens grown on an agar plate, such as a Potato Dextrose Agar (PDA) plate, yet fails to provide adequate effective control of the same pathogens growing on a plant, on fruit, or other plant parts, in the field, or post-harvest. Similarly, even in cases where a single microbial strain exhibits effective biocontrol, the single microbial strain may be unsuitable for practical or commercial application because it cannot be feasibly cultured to economically attractive, high concentrations of viable cells in fermentation processes, e.g. to at least 1×10⁹, 1×10¹⁰ or 1×10⁻¹¹ CFU/mL.

Because a single microbial strain may not be adequate to accomplish any or all of the aforementioned purposes, disclosed herein are biocontrol compositions comprising more than a single microbial strain. Disclosed herein are methods and compositions generated therefrom related to co-culturing the bacterial strain Gluconobacter cerinus (16S SEQ ID NO: 1) together with the yeast strain Hanseniaspora uvarum (ITS SEQ ID NO: 2), provide several significantly advantageous technical effects relative to the performance of each strain cultured separately, or blends of the two strains cultured separated and subsequently combined in different ratios. These surprising advantages may not have been predicted based on any prior knowledge or subsequent experimental demonstration of each strain cultured separately.

Alternatively, or additionally, a single microbial strain may be unsuitable for practical or commercial application because during storage at ambient or refrigerated conditions for at least 7 days, at least 28 days, or at least 90 days, formulated in liquid suspension or in dried, granulated, encapsulated or other solid form, the single microbial strain it does not retain economically attractive, high absolute concentrations of viable cells in fermentation processes, e.g., to at least 1×10⁹ CFU/mL or more, at least 1×10¹⁰ CFU/mL or more, at least 1×10¹¹ CFU/mL or more, or at least 1×10¹² CFU/mL or more, or because the single microbial strain does not retain, after formulation in liquid suspension or in dried, granulated, encapsulated or other solid form, at least 50% of the initial concentration of viable cells as measured just prior to formulation.

The biocontrol compositions described herein can have anti-fungal activity against fungi of agricultural importance and can be formulated to be used at various points in the production process. For example, these biocontrol compositions can be formulated for use prior to harvest, such as for example incorporating the composition into an irrigation line, foliar spray system, root dip, or administration in combination with a fertilizer, as well as post-harvest during processing, packaging, transportation, storage, and commercial display of the produce, such as for example spraying the harvested produce with the composition or application of the composition to a packaging material used to store or ship the produce. Furthermore, these biocontrol compositions can show improved efficacy when compared to commercial biocontrol compositions.

As used herein, the term “co-culture”, “co-cultured” or “co-culturing” generally refers to growing two microorganisms together in a culture medium, or growing one microorganism in medium conditioned by the other microorganism. The conditioned medium may or may not include cells.

As used herein, “viable cell count” refers to the colony forming units (“CFU”) per unit volume, e.g., CFU/mL, of a microorganism as measured by standard dilution plating methods.

As used herein “total cell count” refers to the number of cells, without regard to viability, as counted, for example, by hemocytometer.

As used herein, “culturing” or “fermentation” refers to growing microbes in a growth medium, and these terms are used interchangeably herein.

As used herein, the terms “microbes” and “microorganisms” are used interchangeably.

As used herein, “fermentation ratio” refers to the ratio of total cell counts of two microorganisms in a co-cultured composition at the end of fermentation.

As used herein, “product ratio” refers to the ratio of total cell counts of two microorganisms in a co-cultured composition, after storage for a pre-selected period of time. The fermentation ratio is the same as the product ratio when the pre-selected time is the end of fermentation.

As used herein, the term “combined” generally refers to mixing together two or more microorganisms which are grown separately and then mixed after growth. These microorganisms may be grown in the same type or different type of culturing apparatus, growth media or fermentation processes. The microorganisms may be left in the culturing media or re-suspended in fresh or different culture media prior to combining the microorganisms.

As used herein, the term “strawberry fruit” refers to the whole fruit of a strawberry including the berry and any attached leaves or stems remaining post-harvest.

As used herein, the term “fungal disease incidence”, herein abbreviated as FDI, refers to the appearance of fungal growth on a fruit.

As used herein, the term “local Botrytis disease incidence”, herein abbreviated as LBDI, refers to the appearance of Botrytis at or near the site on a fruit where the Botrytis is inoculated.

As used herein the term “culturing apparatus” generally refers to a vessel that may be used to grow microbes. For example, a culturing apparatus may be, but not limited to: shake flasks, plates, fermentation tanks, fermentors or bioreactors.

### Compositions for the Prevention or Reduction of Crop Loss and Food Spoilage

Disclosed herein are biocontrol compositions which can prevent or reduce the growth of a fungal pathogen on a plant, a seed, or a produce thereof. The term “produce” can be used herein to refer to the edible portion of a plant, such as for example, the leaves, the stem, the seeds, the root, the flowers or the fruit. The term “plant” can be used herein to refer to any portion of the plant, such as for example the leaves, the stem, the seeds, the root, or the fruit. Preventing or reducing the growth of fungal pathogens on the plant, the seed, or the produce thereof can reduce the amount of crop loss and food spoilage prior to, during, or after harvesting the produce from the plant. The biocontrol composition may comprise at least one microbe. Table 1 illustrates the microbial strain identifiers, putative microbial genus or species, and corresponding SEQ ID NOs listed in Table 2. The at least one microbe can be a microbe listed in Table 1.

TABLE 1
Microbial strains with anti-fungal activity

| Microbial strain | Putative microbial genus or | | 16S or |
|---|---|---|---|
| identifier(s) | species | SEQ ID NO. | ITS |
| BC18 | Gluconobacter cerinus | SEQ ID NO: 1 | 16S |
| BC18 | Hanseniaspora uvarum | SEQ ID NO: 2 | ITS |

TABLE 2
Sequences

| SEQ ID NO | Sequence |
|---|---|
| SEQ ID NO: 1 | CGAAGGGGGCTAGCGTTGCTCGGAATGACTGGGCGTA |
| | AAGGGCGCGTAGGCGGTTTATGCAGTCAGATGTGAAA |
| | TCCCCGGGCTTAACCTGGGAACTGCATTTGAGACGCA |
| | TAGACTAGAGGTCGAGAGAGGGTTGTGGAATTCCCAG |
| | TGTAGAGGTGAAATTCGTAGATATTGGGAAGAACACC |
| | GGTGGCGAAGGCGGCAACCTGGCTCGATACTGACGCT |
| | GAGGCGCGAAAGCGTGGGGAGCAAACAG |
| SEQ ID NO: 2 | AGTCGTAACAAGGTTTCCGTAGGTGAACCTGCGGAAG |
| | GATCATTAGATTGAATTATCATTGTTGCTCGAGTTCT |
| | TGTTTAGATCTTTTACAATAATGTGTATCTTTATTGA |
| | AGATGTGCGCTTAATTGCGCTGCTTCTTTAAAGTGTC |
| | GCAGTGAAAGTAGTCTTGCTTGAATCTCAGTCAACGC |
| | TACACACATTGGAGTTTTTTTACTTTAATTTAATTCT |
| | TTCTGCTTTGAATCGAAAGGTTCAAGGCAAAAAACAA |
| | ACACAAACAATTTTATTTTATTATAATTTTTTAAACT |
| | AAACCAAAATTCCTAACGGAAATTTTAAAATAATTTA |
| | AAACTTTCAACAACGGATCTCTTGGTTCTCT |

The at least one microbe may be at least two microbes. The at least two microbes can comprise a first microbe being a Gluconobacter species and a second microbe being a Hanseniaspora species. The at least two microbes can comprise a first microbe being a Gluconobacter cerinus and a second microbe being a Hanseniaspora uvarum.

The at least two microbes can comprise a first microbe with a 16S sequence greater than 90% identical to SEQ ID NO: 1 and a second microbe with a ITS sequence greater than 90% identical to SEQ ID NO: 2. The at least two microbes can comprise a first microbe with a 16S sequence greater than 95% identical to SEQ ID NO: 1 and a second microbe with a ITS sequence greater than 95% identical to SEQ ID NO: 1. The at least two microbes can comprise a first microbe with a 16S sequence greater than 98% identical to SEQ ID NO: 1 and a second microbe with a ITS sequence greater than 98% identical to SEQ ID NO: 2.

In one embodiment, the at least one microbe comprises at least one microbe with at least about: 85%, 87%, 90%, 92%, 95%, 96%, 97%, 98%, 99%, 99.5%, or 100% sequence identity to a rRNA sequence from a Gluconobacter species. The Gluconobacter species can be Gluconobacter cerinus. The rRNA sequence can be a 16S sequence. In one embodiment, the at least one microbe comprises at least one microbe with at least about: 85%, 87%, 90%, 92%, 95%, 96%, 97%, 98%, 99%, 99.5%, or 100% sequence identity to SEQ ID NO: 1.

In one embodiment, the at least one microbe comprises at least one microbe with at least about: 85%, 87%, 90%, 92%, 95%, 96%, 97%, 98%, 99%, 99.5%, or 100% sequence identity to an rRNA sequence from a Hanseniaspora species. The Hanseniaspora species can be Hanseniaspora uvarum. The rRNA sequence can be an ITS sequence. In one embodiment, the at least one microbe comprises at least one microbe with at least about: 85%, 87%, 90%, 92%, 95%, 96%, 97%, 98%, 99%, 99.5%, or 100% sequence identity to SEQ ID NO: 2. In one embodiment, the at least one microbe comprises at least one microbe with at least 90% sequence identity to SEQ ID NO: 2. In one embodiment, the at least one microbe comprises at least one microbe with at least 95% sequence identity to SEQ ID NO: 2. In one embodiment, the at least one microbe comprises at least one microbe with at least 99% sequence identity to SEQ ID NO: 2.

The at least one microbe can be grown in a culture. The at least one microbe can be isolated and purified from the culture. The at least one microbe purified from the culture can comprise a vegetative cell or spore of the at least one microbe. The culture can be a solid or semi-solid medium. The culture can be a liquid medium.

A culture can be a grown in a culturing apparatus. A culturing apparatus can be a bioreactor. Any suitable bioreactor can be used. Examples of bioreactors include, but are not limited to a flask, continuously stirred tank bioreactor (CSTR), a bubbleless bioreactor, an airlift reactor, and a membrane bioreactor. The culturing apparatus may be a particular size or volume to facilitate fermentation at any of a range of scales. For example, the culturing apparatus may be a 3 liter culturing apparatus. In another example, the culturing apparatus may be a 14 liter apparatus. The culturing apparatus may be larger than 0.1, 0.2, 0.3, 0.4, 0.5, 0.6, 0.7, 0.7, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 500000, or 1000000, or more liters in volume. The culturing apparatus may no larger than 0.1, 0.2, 0.3, 0.4, 0.5, 0.6, 0.7, 0.7, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 500000, or 1000000 liters in volume.

The culture may be grown to a high concentration of cells in a particular size or volume of culturing apparatus. For example, the concentration of viable cells may be at least 1×10⁹, 1×10¹⁰, or 1×10¹¹ in a particular size or volume of culturing apparatus.

In some instances, a supernatant of the culture comprises a secondary metabolite of the least one microbe. The secondary metabolite of the at least one microbe can be isolated and purified from the supernatant. In some cases, the supernatant can be applied as the biocontrol composition as described elsewhere herein.

The biocontrol composition can comprise one or more secondary metabolites of the at least one microbe. The one or more secondary metabolites can have antifungal properties of its own, apart from the at least one microbe. The one or more secondary metabolites may with other microbes in a biocontrol composition have antifungal properties. The one or more secondary metabolites can be isolated from a supernatant of the culture of the at least one microbe. The one or more secondary metabolites can comprise a lipopeptide, a dipeptide, an aminopolyol, a polypeptide, a protein, a siderophore, a phenazine compound, a polyketide, or a combination thereof.

The one or more secondary metabolites can comprise a lipopeptide. The lipopeptide can be a linear lipopeptide or a cyclic lipopeptide (CLP). Examples of lipopeptides include, but are not limited to a surfactin, a fengycin, an iturin, a massetolide, an amphisin, an arthrofactin, a tolassin, a syringopeptide, a syringomycin, a putisolvin, a bacillomycin, a bacillopeptin, a bacitracin, a polymyxin, a daptomycin, a mycosubtilin, a kurstakin, a tensin, a plipastatin, a viscosin, and an echinocandin. The echinocandin can be echinocandib B (ECB). In some instances, the secondary metabolite is a surfatin, a fengycin, an iturin, or a combination thereof.

The one or more secondary metabolites can comprise a dipeptide. The dipeptide can be bacilysin or chlorotetain. The polyketide can be defficidin, macrolactin, bacillaene, butyrolactol A, soraphen A, hippolachnin A, or forazoline A. The secondary metabolite can be an aminopolyol. The aminopolyol can be zwittermicin A. The secondary metabolite can be a protein. The protein can be a bacisubin, subtilin, or a fungicin.

The one or more secondary metabolites can comprise a siderophore. The siderophore can be a pyoverdine, thioquinolobactin, or a pyochelin.

The one or more secondary metabolites can comprise a phenazine. The phenazine compound can be a phenzine-1-carboxylic acid, a 1-hydroxyphenazine, or a phenazine-1-carboxaminde.

The secondary metabolite can be a chitinase, a cellulase, an amylase, or a glucanase. The secondary metabolite can be a volatile antifungal compound. The secondary metabolite can be an organic volatile antifungal compound.

As disclosed herein, the biocontrol composition of the present disclosure can be formulated as a liquid formulation or a dry formulation. The liquid formulation can be a flowable or an aqueous suspension. The liquid formulation can comprise the at least one microbe or a secondary metabolite thereof suspended in water, oil, or a combination thereof (an emulsion). The biocontrol composition may be formulated such that the liquid formulation does not comprise precipitates or phase separation. A dry formulation can be a wettable powder, a dry flake, a dust, or a granule. A wettable powder can be applied to the plant, the seed, the flower, or the produce thereof as a suspension. A dust can be applied to the plant, the seed, or the produce thereof dry, such as to seeds or foliage. A granule can be applied dry or can be mixed with water to create a suspension or dissolved to create a solution. The at least one microbe or a secondary metabolite thereof can be formulated as a microencapsulation, wherein the at least one microbe or a secondary metabolite thereof has a protective inert layer. The protective inert layer can comprise any suitable polymer.

The biocontrol composition can further comprise an additional compound. The additional compound can be a carrier, a surfactant, a wetting agent, a penetrant, an emulsifier, a spreader, a sticker, a stabilizer, a nutrient, a binder, a desiccant, a thickener, a dispersant, a UV protectant, or a combination thereof. The carrier can be a liquid carrier, a mineral carrier, or an organic carrier. Examples of a liquid carrier include, but are not limited to, vegetable oil or water. Examples of a mineral carrier include, but are not limited to, kaolinite clay or diatomaceous earth. Examples of an organic carrier include, but are not limited to, grain flour. The surfactant can be an anionic surfactant, a cationic surfactant, an amphoteric surfactant, or a nonionic surfactant. The surfactant can be Tween 20 or Tween 80. The wetting agent can comprise a polyoxyethylene ester, an ethoxy sulfate, or a derivative thereof. In some cases a wetting agent is mixed with a nonionic surfactant. A penetrant can comprise a hydrocarbon. A spreader can comprise a fatty acid, a latex, an aliphatic alcohol, a crop oil (e.g. cottonseed), or an inorganic oil. A sticker can comprise emulsified polyethylene, a polymerized resin, a fatty acid, a petroleum distillate, or pregelantinized corn flour. The oil can be coconut oil, palm oil, castor oil, or lanolin. The stabilizer can be lactose or sodium benzoate. The nutrient can be molasses or peptone. The binder can be gum arabic or carboxymethylcellulose. The desiccant can be silica gel or an anhydrous salt. A thickener can comprise a polyacrylamide, a polyethylene polymer, a polysaccharide, xanthan gum, or a vegetable oil. The dispersant can be microcrystalline cellulose. The UV protectant can be oxybenzone, Blankophor BBH, or lignin.

The biocontrol composition can further comprise dipicolinic acid.

The at least one microbe can comprise an effective amount of isolated and purified microbes isolated and purified from a liquid culture. The at least one microbe from the liquid culture can be air-dried, freeze-dried, spray-dried, or fluidized bed-dried to produce a dry formulation. The dry formulation can be reconstituted in a liquid to produce a liquid formulation.

The biocontrol composition can be formulated such that the at least one microbe can replicate once they are applied/or delivered to the target habitat (e.g. the soil, the plant, the seed, and/or the produce).

The biocontrol composition can have a shelf life of at least one week, one month, six months, at least one year, at least two years, at least three years, at least four years, or at least five years. The shelf life can indicate the length of time the biocontrol composition maintains at least 50%, at least 55%, at least 60%, at least 65%, at least 70%, at least 75%, at least 80%, at least 85%, at least 90%, at least 95%, at least 99%, or 100% of its anti-fungal properties. The biocontrol composition can be stored at room temperate, at or below 10° C., at or below 4° C., at or below 0° C., or at or below −20° C. The biocontrol composition may be formulated to retain viability of the at least one microbe. The biocontrol composition may be formulated such that the cfu/ml (colony forming units per milliliter) after being stored for a time period is not substantially reduced. This may be relative to a biocontrol composition that is not formulated, or relative to a biocontrol composition which is not co-cultured (e.g., cultured alone and then individually combined) as disclosed herein. For example, the cfu/ml of a formulated biocontrol composition may be reduced by no more than 10 times (e.g., 1 log) after being stored for 4 weeks at 25° C. For example, the cfu/ml of a formulated biocontrol composition may be reduced by no more than 2, 3, 4, 5, 6, 7, 8, 9, 10, 11, 12, 13, 14, 15, 16, 17, 18, 19, 20, 25, 30, 35, 40, 45, 50, 100, or 1000 times, after being stored for 4 weeks at 25° C.

The biocontrol composition may retain the viability of the at least one microbe when stored at a variety of temperatures. For example, the cfu/ml of the biocontrol composition may be reduced by no more than 10 times (e.g., 1 log) after being stored at 4 weeks at 0° C. For example, the cfu/ml of the biocontrol composition may be reduced by no more than 10 times after being stored at 4 weeks at 4° C. For example, the cfu/ml of the biocontrol composition may be reduced by no more than 10 times after being stored at 4 weeks at 10° C. For example, the cfu/ml of the biocontrol composition may be reduced by no more than 10 times after being stored at 4 weeks at −20° C. For example, the cfu/ml of the biocontrol composition may be reduced by no more than 10 times after being stored at 4 weeks at −80° C.

The biocontrol composition may retain viability after storage for a given period of time. For example, the cfu/ml of the biocontrol composition may be reduced by no more than 10 times after storage at a given temperature for 1, 2, 3, 4, 5, 6, 7, 8, 9, 10, 15, 20, 25, 30, 40, 50, 60, 70, 80, 90, 100, or more weeks.

The biocontrol composition may be formulated to retain anti-pathogenic activity after storage of a time period. Such pathogenic activity of a stored formulation may be substantially equivalent to a fresh biocontrol composition. An unaged or fresh biocontrol composition may comprise a co-culture obtained from a fermentation apparatus, without being subjected to storage conditions.

The biocontrol composition may be formulated such that the anti-pathogenic activity is not substantially reduced after storage for a time period. For example, the biocontrol composition may be formulated such that the dosage of a stored biocontrol composition applied is no more than 10 times the dosage of a fresh (unaged) biocontrol composition. For example, the biocontrol composition may be formulated such that the dosage of a stored biocontrol composition applied after storage is no more than 1, 2, 3, 4, 5, 6, 7, 8, 9, 10, 11, 12, 13, 14, 15, 16, 17, 18, 19, or 20 times the dosage of a fresh (unaged) biocontrol composition.

A stored biocontrol composition of the present disclosure may be combined with a biostimulant composition prior to application or use. The biostimulant composition may allow the plant to grow at a faster rate than a comparable plant without the biostimulant composition. The biostimulant composition may for example, increase nutrient uptake, nutrient usage efficiency, improve recovery or resilience to abiotic stress, or combinations thereof. Examples of biostimulants include Azospirillum, such as TAZO®-B Microbial Bio-Stimulant, which may increase nitrogen fixation or increase root mass, or Bacillus amyloliquefaciens and Trichoderma vixens based biostimulants such as Novozymes QuickRoots®, which may increase availability or uptake of nitrogen, phosphate or potassium. Post-storage, the biocontrol composition may have a retained viability such that the number of viable microbes (cfu/mL) provides a sufficient degree of anti-fungal activity (e.g., against Botrytis cinerea).

As described elsewhere herein, the biocontrol composition may be stored at a variety of different temperature and time periods and may still maintain viability of the at least one microbe. Similarly, the anti-pathogenic or anti-fungal activity may be maintained (or reduced by a small factor) after storage. For example, after storage for 4 weeks at 25° C., the dosage used to inhibit fungal growth may be no more than 10 times the dosage of a fresh (unaged) biocontrol composition. For example, a dosage used to inhibit fungal growth may be no more than 2, 3, 4, 5, 6, 7, 8, 9, 10, 11, 12, 13, 14, 15, 16, 17, 18, 19, 20, 25, 30, 35, 40, 45, 50, 100, or 1000 times the dosage of a fresh (unaged) biocontrol composition after storage at up to 4 weeks at 25° C. The biocontrol composition may retain anti-pathogenic or anti-fungal activity when stored at a variety of temperatures. For example, the dosage used to inhibit fungal growth may be no more than 10 times the dosage of a fresh (unaged) biocontrol composition after storage for up to 4 weeks at 0° C. In another example, the dosage used to inhibit fungal growth may be no more than 10 times the dosage of a fresh (unaged) biocontrol composition after 4 weeks at 4° C. The dosage used to inhibit fungal growth may be no more than 10 times the dosage of a fresh (unaged) biocontrol composition after 4 weeks at 10° C. The dosage used to inhibit fungal growth may be no more than 10 times the dosage of a fresh (unaged) biocontrol composition after 4 weeks at −20° C. For example, the dosage used to inhibit fungal growth may be no more than 10 times the dosage of a fresh (unaged) biocontrol composition after 4 weeks at −80° C.

The biocontrol composition may retain anti-pathogenic or anti-fungal activity after storage for a given period of time. For example, the dosage used to inhibit fungal growth may be no more than 10 times the dosage of a fresh (unaged) biocontrol composition after storage at a given temperature for 1, 2, 3, 4, 5, 6, 7, 8, 9, 10, 15, 20, 25, 30, 40, 50 or more weeks.

The biocontrol composition can comprise spores. Spore-containing compositions can be applied by methods described herein. Spore-containing compositions can extend the shelf life of the biocontrol composition. Spore-containing compositions can survive low pH or low temperatures of a target habitat. For example, spore-containing compositions may be applied to the soil at a colder temperature (for example, below 10° C.) and can have anti-fungal properties for a seed planted at a higher temperature (for example, 20° C.). The spores may become vegetative cells, allowing them any advantages of vegetative cells.

The biocontrol composition can comprise vegetative cells. Vegetative cell-containing compositions can be applied by methods described herein. Vegetative cells may proliferate and increase efficacy of the composition. For example, vegetative cells in the biocontrol composition may proliferate after application increasing the surface area of the plant that is exposed to the biocontrol composition. In another example, vegetative cells in the biocontrol composition may proliferate after application increasing the amount of the time the biocontrol composition survives and thus extending the time the biocontrol composition has efficacy. The vegetative cells may proliferate and compete for nutrients with a fungal pathogen. The vegetative cells may actively produce one or more secondary metabolites with anti-fungal properties. The vegetative cells may become spores, allowing them any advantages of spores.

The biocontrol composition can have anti-fungal activity, such as prevention of growth of a fungal pathogen or reduction of growth of a fungal pathogen on a plant, a seed, or a produce thereof. The biocontrol composition can prevent growth of a fungal pathogen on the plant, seed, or produce thereof for at least 1, at least 2, at least 3, at least 4, or at least 5 days. The biocontrol composition can prevent growth of a fungal pathogen on the plant, seed, or produce thereof for at least 1, at least 2, at least 3, at least 4, at least 5 days, at least 6 days, at least 7 days, at least 8 days, at least 9 days, or at least 10 days. The biocontrol composition can prevent growth of a fungal pathogen on the plant, seed, or produce thereof for over 10 days.

The biocontrol composition can reduce growth of the fungal pathogen on the plant, seed, or produce thereof relative to growth of the fungal pathogen on a control that is a plant, a seed, flower, or a produce thereof not exposed to the biocontrol composition. The control can be a plant, a seed, or a produce thereof to which no anti-fungal agent has been applied or can be a plant, a seed, flower, or produce thereof to which a commercially available anti-fungal agent has been applied. Examples of commercially available anti-fungal agents include, but are not limited to, Bacillus subtilis strain QST713 (Serenade®), Bacillus subtilis strain GB02 (Kodiak®), Bacillus subtilis strain MBI 600 (Subtilex®), Bacillus pumilus strain GB34 (YieldShield), Bacillus licheniformis strain SB3086 (EcoGuard®). The biocontrol composition can reduce growth of a fungal pathogen on the plant, seed, or produce thereof for at least 1, at least 2, at least 3, at least 4, or at least 5 days. The biocontrol composition can reduce growth of a fungal pathogen on the plant, seed, or produce thereof for at least 1, at least 2, at least 3, at least 4, at least 5 days, at least 6 days, at least 7 days, at least 8 days, at least 9 days, or at least 10 days. The biocontrol composition can reduce growth of a fungal pathogen on the plant, seed, or produce thereof for over 10 days. The biocontrol composition can reduce growth of the fungal pathogen of at least 25% relative to growth of the fungal pathogen on the control. The biocontrol composition can reduce growth of the fungal pathogen of at least 60% relative to growth of the fungal pathogen on the control. The biocontrol composition can reduce growth of the fungal pathogen of at least 25%, 30%, 35%, 40%, 45%, 50%, 55%, 60%, 65%, 70%, 75%, 80%, 85%, 90%, 95%, 99%, or more relative to growth of the fungal pathogen on the control.

The fungal pathogen can be a fungal pathogen in the genus Albugo, Alternaria, Aphanomyces, Armillaria, Aspergillus, Botrytis, Botrydiplodia, Botrytinia, Bremia, Cercospora, Cercosporella, Cladosporium, Colletotrichum, Cordana, Corynespora, Cylindrocarpon, Daktulosphaira, Didymella, Elsinoe, Erysiphe, Eutypa, Fusarium, Gaeumannomyce, Ganoderma, Geotrichum, Guignardia, Gymnoconia, Helminthosporium, Leptosphaeria, Leveillula, Macrophomina, Microsphaera, Monolinia, Mycosphaerella, Oidopsis, Passalora, Penicillium, Peronospora, Phomopsis, Phytophthora, Peronospora, Pestalotiopsis, Phoma, Plasmodiophora, Plasmopara, Podosphaera, Polyscytalum, Pseudocercospora, Puccinia, Pucciniastrum, Pythium, Ralstonia, Ramularia, Rhizoctonia, Rhizopus, Septoria, Sclerotinia, Sclerotium, Sphaerotheca, Sphaceloma, Spongospora, Stemphylium, Synchytrium, Thielaviopsis, Uncinula, Uromyces, or Verticillium. The fungal pathogen can be Albugo candida, Albugo occidentalis, Alternaria alternata, Alternaria cucumerina, Alternaria dauci, Alternaria solani Alternaria tenuis, Alternaria tenuissima, Alternaria tomatophila, Aphanomyces euteiches, Aphanomyces raphani, Armillaria mellea Aspergillus flavus, Aspergillus parasiticus, Botrydia theobromae, Botrytis cinerea, Botrytinia fuckeliana, Bremia lactuca, Cercospora beticola, Cercosporella rubi, Cladosporium herbarum, Colletotrichum acutatum, Colletotrichum gloeosporioides, Colletotrichum lindemuthianum, Colletotrichum musae, Colletotrichum spaethanium, Cordana musae, Corynespora cassiicola, Daktulosphaira vitifoliae, Didymella bryoniae, Elsinoe ampelina, Elsinoe mangiferae, Elsinoe veneta, Erysiphe cichoracearum, Erysiphe necator, Eutypa lata, Fusarium germinareum, Fusarium oxysporum, Fusarium solani, Fusarium virguliforme, Gaeumannomyces graminis, Ganoderma boninense, Geotrichum candidum, Guignardia bidwellii, Gymnoconia peckiana, Helminthosporium solani, Leptosphaeria coniothyrium, Leptosphaeria maculans, Leveillula taurica, Macrophomina phaseolina, Microsphaera alni, Monilinia fructicola, Monilinia vaccinii-corymbosi, Mycosphaerella angulate, Mycosphaerella brassicicola, Mycosphaerella fragariae, Mycosphaerella fijiensis, Oidopsis taurica, Passalora Alva, Penicillium expansum, Peronospora sparse, Peronospora farinosa, Pestalotiopsis clavispora, Phoma exigua, Phomopsis obscurans, Phomopsis vaccinia, Phomopsis viticola, Phytophthora capsica, Phytophthora erythroseptica, Phytophthora infestans, Phytophthora parasitica, Phytophthora ramorum, Plasmopara viticola, Plasmodiophora brassicae, Podosphaera macularis, Polyscytalum pustulans, Pseudocercospora vitis, Puccinia allii, Puccinia sorghi, Pucciniastrum vaccinia, Pythium aphanidermatum, Pythium debaryanum, Pythium sulcatum, Pythium ultimum, Ralstonia solanacearum, Ramularia tulasneii, Rhizoctonia solani, Rhizopus arrhizus, Rhizopus stoloniferz, Sclerotinia minor, Sclerotinia homeocarpa, Sclerotium cepivorum, Sclerotium rolfsii, Sclerotinia minor, Sclerotinia sclerotiorum, Septoria apiicola, Septoria lactucae, Septoria lycopersici, Septoria petroelini, Sphaceloma perseae, Sphaerotheca macularis, Spongospora subterrannea, Stemphylium vesicarium, Synchytrium endobioticum, Thielaviopsis basicola, Uncinula necator, Uromyces appendiculatus, Uromyces betae, Verticillium albo-atrum, Verticillium dahliae, Verticillium theobromae, or a combination thereof. The fungal pathogen can be Fusarium oxysporum or Verticillium dahliae. The fungal pathogen can be Botrytis cinerea. The fungal pathogen can be Colletotrichum spaethanium. The fungal pathogen can be Erysiphe necator. The fungal pathogen can be Peronospora farinosa. The fungal pathogen can be Podosphaera maculari. The fungal pathogen can be Monilinia vaccinii-corymbosi. The fungal pathogen can be Puccinia sorghi. The fungal pathogen may be Penicillium expansum. The fungal pathogen can be a fungal pathogen causing Powdery Mildew. The fungal pathogen can be a fungal pathogen causing Downy Mildew. The fungal pathogen can be a fungal pathogen causing mummy berry. The fungal pathogen can be a fungal pathogen causing corn rust.

The plant, flower, seed, or produce thereof can be of an almond, apricot, apple, artichoke, banana, barley, beet, blackberry, blueberry, broccoli, Brussels sprout, cabbage, cannabis, canola, capsicum, carrot, celery, chard, cherry, citrus, corn, cotton, cucurbit, date, fig, flax, garlic, grape, herb, spice, kale, lettuce, mint, oil palm, olive, onion, pea, pear, peach, peanut, papaya, parsnip, pecan, persimmon, plum, pomegranate, potato, quince, radish, raspberry, rose, rice, sloe, sorghum, soybean, spinach, strawberry, sweet potato, tobacco, tomato, turnip greens, walnut, or wheat. The plant, seed, flower, or produce thereof can be a plant or produce thereof can be from the family Rosaceae. The plant, flower, seed, or produce thereof from the family Rosaceae can be from the genus Rubus, such as a raspberry or blackberry, Fragaria, such as a strawberry, Pyrus such as a pear, Cydonia such as a quince, Prunus, such as an almond, peach, plum, apricot, cherry or sloe, Rosa, such as a rose, or Malus, such as an apple. The plant, seed, flower, or produce thereof can be a plant or produce thereof from the family Ericaceae. The plant, seed, flower, or produce thereof from the family Ericaceae can be from the genus Vaccinium, such as a blueberry. The plant, seed, flower, or produce thereof can be a plant or produce thereof from the family Ericaceae. The plant, seed, flower, or produce thereof from the family Ericaceae can be from the genus Vaccinium, such as a blueberry. The plant, seed, flower, or produce thereof can be a plant or produce thereof from the family Vitaceae. The plant, seed, flower, or produce thereof from the family Vitaceae can be from the genus Vitis, such as a grape.

### Methods of Identification and Isolation of the Biocontrol Composition.

Methods of identifying and/or selecting for a biocontrol composition can comprise culturing the at least one microbe in isolation or with a plurality of other microbes and/or fungal pathogens. For example, the at least one microbe can be cultured with a fungal pathogen to identify efficacy of the at least one microbe to inhibit growth of the fungal pathogen. The efficacy of the at least one microbe to inhibit the growth of the fungal pathogen can be determined by observing the growth parameters of the fungal pathogen. For example, the lack of living fungal pathogen close to the at least one microbe on a semi-solid or solid growth media may be used determine a high efficacy of inhibition. The optical density of a liquid media containing the at least one microbe and the fungal pathogen may be used to identify an efficacy of the at least one microbe.

The at least one microbe can be identified by a variety of methods. The at least one microbe can be subjected to a sequencing reaction. The sequencing reaction may identify a sequence of 16S rRNA, 12S rRNA, 18S rRNA, 28S rRNA, 13S rRNA and 23S rRNA, internal transcribed spacer (ITS), ITS1, ITS2, cytochrome oxidase I (COI), cytochrome b, or any combination thereof. The sequencing reaction may identify a 16S rRNA sequence, an ITS sequence, or a combination thereof. The sequencing reaction and sequencing reads generated therefrom may be used to identify the species or strain of the at least one microbe. Sequencing reads generated from sequencing reaction(s) may be processed against one or more reference sequences to facilitate the identification of the at least one microbe.

The at least one microbe may be affected by other microbes. The microbes can behave synergistically when cultured together such that the anti-fungal properties are improved when cultured together compared to when cultured separately. For example, the at least one microbe may have increased viability when cultured with another microbe. The at least one microbe may have increased proliferation when cultured with another microbe. The at least one microbe may use chemicals or metabolites produced by another microbe. The at least one microbe may interact directly with another microbe. For example, the at least one microbe and another microbe may form biofilms or a multicellular structure. The at least one microbe may produce and/or secrete an increased amount of the secondary metabolite when cultured with another microbe. For example, the at least one microbe may produce an intermediate metabolite, which in turn is processed by another microbe resulting in the secondary metabolite. Methods disclosed elsewhere herein can be used to identify microbes which may benefit from culturing with another microbe, as well as identify biocontrol compositions comprising a first microbe and a second microbe, wherein the second microbe is not identical to the first microbe.

Co-culturing microbes may be performed in a variety of manners that allow multiple microbes to interact or grow together. For example, a first microbe may be cultured and a second microbe can then be combined with the first microbe culture, or vice versa. Gluconobacter cerinus may be the first microbe and Hanseniaspora uvarum may be the second microbe. Alternatively, Hanseniaspora uvarum may be the first microbe and Gluconobacter cerinus may be the second microbe. In another non-limiting example, the first microbe may be cultured in a first culturing apparatus and the second microbe may be cultured in a second culturing apparatus prior to combining the first microbe and second microbe. The first microbe may then be moved from the first culturing apparatus to the second culturing apparatus, thereby combining the first and second microbe in a single culturing apparatus. In some cases, the movement of the first microbe to the second culturing apparatus may be facilitated by centrifugation, and resuspension. For example, the first microbe may be pelleted using the centrifuge, resuspended in a new liquid and then added to the second apparatus. In some cases, the media containing the first microbe can be poured directly into the second culturing apparatus. The second microbe could be subjected to centrifugation and the media containing the first microbe may be added to the second culturing apparatus. The first and second microbe could be directly inoculated in a single culturing apparatus. The first microbe may be directly inoculated in a culture that already contains the second microbe. The two microbes may be introduced into a co-culture in any order. For example, the first microbe may be introduced to a culture followed by the second, or the second microbe may be introduced to a culture followed by the first. The first and second microbes may be introduced simultaneously or substantially simultaneously to a culture. Co-culturing may comprise growing one microbe in medium conditioned by the other microbe. The conditioned medium may or may not include cells. For example, a first microbe may be grown in a first media and then may be removed from the first media. A second microbe may then be introduced into the first media and allowed to proliferate.

As described above co-culturing may be performed in a culturing apparatus. In addition to the culturing apparatus, co-cultures may be directly generated on the plant, flower, seed, or produce thereof. Co-cultures may be generated directly on the packaging in which the plant, flower, seed, or produce thereof is packaged or otherwise stored in. As disclosed elsewhere herein each microbe in the co-culture may be applied to the plant, flower, seed, or produce thereof, or packaging in various orders and amounts to generate the co-culture.

The biocontrol composition may comprise the at least two microbe in specific product ratios of amounts of each microbe. For example, the first and second microbe may be in a 1:1 product ratio. The first and second microbes may be in a 1:3 product ratio. The first and second microbes may be in a 3:1 product ratio. The first and second microbes may be in a product ratio, wherein the amount of the first microbe compared to the second microbe is a least in 1:1, 1:2, 1:3, 1:4, 1:5, 1:6, 1:7, 1:8, 1:9, 1:10, 1:11, 1:12, 1:13, 1:14, 1:15, 1:16, 1:17, 1:18; 1:19: 1:20, 1:25, 1:30, 1:35, 1:40, 1:50, 1:60, 1:70, 1:80, 1:90, or 1:100, or more. The first and second microbes may be in a product ratio, wherein the amount of the first microbe compared to the second microbe is at least 1:1, 2:1, 3:1, 4:1, 5:1, 6:1, 7:1, 8:1, 9:1, 10:1, 11:1, 12:1, 13:1, 14:1, 15:1, 16:1, 17:1, 18:1; 19:1: 20:1, 25:1, 30:1, 35:1, 40:1, 50:1, 60:1, 70:1, 80:1, 90:1, or 100:1, or more. The first and second microbe may be present in a range of product ratios from 1:1 to 1:100 or 1:1 to 1:10. The first and second microbe may be present in a range of product ratios from 1:1 to 100:1 or 1:1 to 10:1. The first and second microbe may be present in a range of product ratios from 100:1 to 1:100 or 10:1 to 1:10. The first and second microbes may be in a product ratio, wherein the amount of the first microbe compared to the second is a no more than in 1:1, 1:2, 1:3, 1:4, 1:5, 1:6, 1:7, 1:8, 1:9, 1:10, 1:11, 1:12, 1:13, 1:14, 1:15, 1:16, 1:17, 1:18; 1:19: 1:20, 1:25, 1:30, 1:35, 1:40, 1:50, 1:60, 1:70, 1:80, 1:90, or 1:100, or less. The first and second microbes may be in a product ratio, wherein the amount of the first microbe compared to the second microbe is no more than 1:1, 2:1, 3:1, 4:1, 5:1, 6:1, 7:1, 8:1, 9:1, 10:1, 11:1, 12:1, 13:1, 14:1, 15:1, 16:1, 17:1, 18:1; 19:1: 20:1, 25:1, 30:1, 35:1, 40:1, 50:1, 60:1, 70:1, 80:1, 90:1, or 100:1, or less. In a non-limiting example, the first microbe may be Gluconobacter cerinus and the second microbe may be Hanseniaspora uvarum, and the product ratio of the Gluconobacter cerinus and the Hanseniaspora uvarum may be between about 1:100 and 100:1. In a further non-limiting example, the first microbe may be Gluconobacter cerinus and the second microbe may be Hanseniaspora uvarum, and the product ratio of the Gluconobacter cerinus and the Hanseniaspora uvarum may be between about 1:10 and 10:1. For example, the first microbe may be Gluconobacter cerinus and the second microbe may be Hanseniaspora uvarum, and the product ratio of the Gluconobacter cerinus and the Hanseniaspora uvarum may be about 100:1, 50:1, 20:1, 10:1, 5:1, 3:1, 2:1, 1:1, 1:2, 1:3, 1:5, 1:10, 1:20, 1:50 or 1:100.

In compositions comprising the co-cultured Gluconobacter cerinus and Hanseniaspora uvarum, the co-cultured microbes may have improved activity of reducing or preventing pathogen growth compared to the individual microbes cultured alone, individually or combined after being cultured alone. For example, the composition of the co-cultured Gluconobacter cerinus and Hanseniaspora uvarum may be capable of inhibiting growth of a fungal microorganism 10% or more relative to a reference composition comprising either of the Gluconobacter cerinus and the Hanseniaspora uvarum cultured individually or to the two microorganisms combined at about the same cell density and cell ratio as that of the co-cultured composition. The composition of the co-cultured Gluconobacter cerinus and Hanseniaspora uvarum may be capable of inhibiting growth of a fungal microorganism at least, 5,%, 10%, 15%, 20%, 25%, 30%, 35%, 40%, 45%, 50%, 55%, 60%, 70%, 75%, 80%, 85%, 90%, 95%, or even 100%, relative to a composition comprising either of the at least two microorganisms cultured individually or to the two microorganisms combined at about the same cell density and cell ratio as that of the composition. For example, the composition of the co-cultured Gluconobacter cerinus and Hanseniaspora uvarum may be capable of inhibiting fungal disease incidence of a fungal microorganism 10% or more relative to a reference composition comprising either of the two microorganisms cultured individually or to the two microorganisms combined at about the same cell density and cell ratio as that of the composition. The composition of the co-cultured Gluconobacter cerinus and Hanseniaspora uvarum may be capable of improving fungal disease incidence (FDI) by at least, 5,%, 10%, 15%, 20%, 25%, 30%, 35%, 40%, 45%, 50%, 55%, 60%, 70%, 75%, 80%, 85%, 90%, 95%, 100%, or more relative to a composition comprising either of the two microorganisms cultured individually or to the two microorganisms combined at about the same cell density and cell ratio as that of the composition

For example, the composition of at least two microbes may be capable of reducing fungal disease severity of a fungal pathogen 10% or more relative to a reference composition comprising either of the at least two microbes cultured individually or to the two microbes combined at the same cell density and cell ratio as that of the composition. The composition of at least two microbes may be capable of inhibiting fungal disease severity at least, 5,%, 10%, 15%, 20%, 25%, 30%, 35%, 40%, 45%, 50%, 55%, 60%, 70%, 75%, 80%, 85%, 90%, 95%, 100%, or more relative to a composition comprising either of the at least two microbes cultured individually or to the two microbes combined at the same cell density and cell ratio as that of the composition.

In compositions comprising the co-cultured Gluconobacter cerinus and Hanseniaspora uvarum, the combination of microbes may have improved viability compared to the individual microbes cultured individually or to the two microorganisms combined at about the same cell density and cell ratio as that of the co-cultured composition. The combination or co-culture of microbes may have a viable cell count at the end of fermentation of the co-cultured microorganisms, grown using a given fermentation medium, feed composition and fermentation process, which is more than five times the sum of the viable cell counts of the individual microorganisms grown alone using the equivalent fermentation medium, feed composition and fermentation process. The co-cultured Gluconobacter cerinus and Hanseniaspora uvarum may have a viable cell count at the end of fermentation, grown using a given fermentation medium, feed composition and process, which is more which is more than 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 50, 60, 70, 80, 90, or 100, or more times the sum of the viable cell counts the individual microorganisms grown alone in the equivalent fermentation medium, feed composition and fermentation process. The co-cultured Gluconobacter cerinus and Hanseniaspora uvarum after fermentation may have a 10%, 20%, 30%, 40%, 50%, 60%, 70%, 80%, 90%, 100%, or more, higher cell density than the cell density of the individual microorganism grown alone in the same fermentation process. For example, the viable cell counts or cell density of the co-cultured microbes may be as high as 10⁹, 10¹⁰, 10¹¹, 10¹² or more CFU/mL.

In compositions comprising the co-cultured Gluconobacter cerinus and Hanseniaspora uvarum, the combination of microbes may have increased viability, even upon storage of the microbe, as compared to that of the individual microbes alone. For example, the viable cell count of the co-cultured Gluconobacter cerinus and Hanseniaspora uvarum after storage at a constant temperature between 4° C. and 25° C., for at least 7 days, is higher than the sum of the viable cell counts of the microbes grown alone in the equivalent fermentation process and subjected to an equivalent storage condition. For example, the viable cell count of the composition after storage at a constant temperature between 4° C. and 25° C., for at least 7 days, is at least 10%, 20%, 30%, 40%, 50%, 60%, 70%, 80%, 90%, 100%, or more, higher than the sum of the viable cell counts of the microbes grown alone in the equivalent fermentation process and subjected to an equivalent storage condition. The composition comprising the co-cultured Gluconobacter cerinus and Hanseniaspora uvarum after storage at a constant temperature between 4° C. and 25° C., for at least 7 days may have a 10%, 20%, 30%, 40%, 50%, 60%, 70%, 80%, 90%, 100%, or more, higher cell density than the cell density of the respective microorganism grown alone in the same fermentation process and subjected to an equivalent storage condition. For example, the cell density may be as high as 10⁹, 10¹⁰ or 10¹¹, 10¹² or more CFU/mL.

In some cases, the co-cultured Gluconobacter cerinus and Hanseniaspora uvarum may be affected by environmental conditions. The co-cultured Gluconobacter cerinus and Hanseniaspora uvarum may grow or produce a secondary metabolite at a particular pH. For example, the pH at which the co-cultured Gluconobacter cerinus and Hanseniaspora uvarum is grown in may be a pH of 3.0, 4.0, 5.0, 6.0, 6.2, 6.4, 6.6, 6.8, 7.0, 7.2, 7.4, 7.6, 7.8, 8.0, 9.0, 10.0 or higher. For example, the pH at which the co-cultured Gluconobacter cerinus and Hanseniaspora uvarum is grown in may be a pH of 3.0, 4.0, 5.0, 6.0, 6.2, 6.4, 6.6, 6.8, 7.0, 7.2, 7.4, 7.6, 7.8, 8.0, 9.0, 10.0 or lower. The co-cultured Gluconobacter cerinus and Hanseniaspora uvarum may grow or produce a secondary metabolite in the presence of salts. The salts may be buffer salts. The co-cultured Gluconobacter cerinus and Hanseniaspora uvarum may grow or produce a secondary metabolite in the presence of sugars or carbohydrates. The sugar or carbohydrate may be glucose or glycerol.

The biocontrol compositions can be cultured using a variety of media or substrate. The co-cultured Gluconobacter cerinus and Hanseniaspora uvarum can be cultures on an agar dish. The co-cultured Gluconobacter cerinus and Hanseniaspora uvarum can be cultured on a semi-solid agar dish. The co-cultured Gluconobacter cerinus and Hanseniaspora uvarum can be cultured in a liquid media.

### Methods for Prevention or Reduction of Food Rot and Food Spoilage

Treating the Plant, the Seed, Flower, or the Produce Thereof with the Biocontrol Composition Prior to Harvest

Methods of preventing or reducing the growth of a fungal pathogen on a plant, a seed, or a produce thereof can comprise applying to the plant, the seed, flower, or the produce, before it has been harvested, a biocontrol composition comprising at least one microbe described herein or one or more secondary metabolites thereof and a carrier. Harvesting the produce can refer to the removal of the edible portion of the plant from the remainder of the plant, or can refer to removal of the entire plant with subsequent removal of the edible portion later.

Applying the biocontrol composition prior to harvest can comprise dusting, injecting, spraying, or brushing the plant, the seed, or the produce thereof with the biocontrol composition. Applying the biocontrol composition can comprise adding the biocontrol composition to a drip line, an irrigation system, a chemigation system, a spray, such as foliar spray, or a dip, such as a root dip. In some cases, the biocontrol composition is applied to the root of the plant, the seed of the plant, the foliage of the plant, the soil surrounding the plant or the edible portion of the plant which is also referred to herein as the produce of the plant.

The method can further comprise applying to the plant a fertilizer, an herbicide, a pesticide, other biocontrols, or a combination thereof. In some instances, the fertilizer, herbicide, pesticide, other biocontrols or combination thereof is applied before, after, or simultaneously with the biocontrol composition.

Methods of preventing or reducing the growth of a fungal pathogen can comprise applying to the seed a biocontrol composition comprising at least one microbe described herein or a secondary metabolite thereof and a carrier. Applying the biocontrol composition to the seed of the plant can occur before planting, during planting, or after planting prior to germination. For example, the biocontrol composition can be applied to the surface of the seed prior to planting. In some cases, a seed treatment occurring before planting can comprise addition of a colorant or dye, a carrier, a binder, a sticker, an anti-foam agent, a lubricant, a nutrient, or a combination thereof to the biocontrol composition.

Methods of preventing or reducing the growth of a fungal pathogen can comprise applying to the soil a biocontrol composition comprising at least one microbe described herein or a secondary metabolite thereof and a carrier. The biocontrol composition can be applied to the soil before, after, or during planting the soil with a seed, or before transfer of the plant to a new site. In one example, a soil amendment is added to the soil prior to planting, wherein the soil amendment results in improved growth of a plant, and wherein the soil amendment comprises the biocontrol composition. In some cases, the soil amendment further comprises a fertilizer.

Methods of preventing or reducing the growth of a fungal pathogen can comprise applying to the root a biocontrol composition comprising at least one microbe described herein or a secondary metabolite thereof and a carrier. The biocontrol composition can be directly applied to the root. One example of a direct application to the root of the plant can comprise dipping the root in a solution that includes the biocontrol composition. The biocontrol composition can be applied to the root indirectly. One example of an indirect application to the root of the plant can comprise spraying the biocontrol composition near the base of the plant, wherein the biocontrol composition permeates the soil to reach the roots.

Treating the Produce Thereof with the Biocontrol Composition after Harvest

Methods of preventing or reducing the growth of a fungal pathogen on a produce can comprise applying to the produce, before or after it has been harvested, a biocontrol composition comprising at least one microbe described herein or a secondary metabolite thereof and a carrier.

Applying the biocontrol composition before or after harvest can comprise dusting, dipping, rolling, injecting, rubbing, spraying, or brushing the produce of the plant with the biocontrol composition. The biocontrol composition can be applied to the produce immediately prior to harvest or immediately after harvesting or within 1 day, 2 days, 3 days, 4 days, 5 days, 6 days, or 1 week of harvesting. In some cases, the biocontrol composition is applied by the entity doing the harvesting, in a process treating the produce immediately prior to harvest or post-harvest, by the entity packaging the produce, by the entity transporting the produce, or by the entity commercially displaying the produce for sale, or a consumer.

Applying the biocontrol composition after harvest can further comprise integrating the biocontrol composition into a process to treat the produce post-harvest. The produce can be treated immediately post-harvest, for example in one or multiple washes. The one or multiple washes can comprise the use of water, or the use of water that has had bleach (chlorine) and/or sodium bicarbonate added to it, or ozonated water. The produce may also be treated with oils, resins, or structural or chemical matrices. The biocontrol composition may be mixed with the oils, resins, or structural or chemical matrices for application. The produce can be treated before or after drying the produce. For example, the biocontrol composition can be added to a wax, gum arabic or other coating used to coat the produce. The biocontrol composition may be added at any point in the process, included in one of the washes, as part of a new wash, or mixed with the wax, gum arabic or other coating of the produce.

Treating a Packaging Material with the Biocontrol Composition

Methods of preventing or reducing the growth of a fungal pathogen on a produce can comprise applying to a packaging material used to transport or store the produce a biocontrol composition comprising at least one microbe described herein or a secondary metabolite thereof and a carrier.

The packaging material can comprise: polyethylene terephthalate (PET), molded fiber, oriented polystyrene (OPS), polystyrene (PS) foam, polypropylene (PP), or a combination thereof. The packaging material can comprise cardboard, solid board, Styrofoam, or molded pulp. The packaging material can comprise a substrate, such as cellulose. The packaging material can be a horizontal flow (HFFS) package, a vertical flow (VFFS) package, a thermoformed package, a sealed tray, or a stretch film. The thermoformed package can be a clam shell package. The packaging material can be a punnet, a tray, a basket, or a clam shell.

The packaging material treated with the biocontrol composition can be an insert. The insert can be a pad, a sheet, or a blanket. The insert can be placed into or over the punnet, the tray, the basket, or the clam shell. The insert can comprise cellulose or a cellulose derivative. The insert can comprise at least one layer of a micro porous polymer such as polyethylene or polypropylene and at least one layer of a superabsorbent polymer. In some cases, the insert comprises an outer layer and an inner layer. The inner layer can be a water-absorbing layer. The inner layer can comprise a carboxymethyl cellulose, cellulose ether, polyvinyl pyrrolidon, starch, dextrose, gelatin, pectin, or a combination thereof. The outer layer can be a water pervious layer.

Applying the biocontrol composition to the packaging material can comprise washing, spraying, or impregnating the packaging material with the biocontrol composition.

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein are. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### EXAMPLES

### Example 1. Co-Cultured BC18 is More Effective Against B. cinerea than BC18 when Recombined into a Consortium

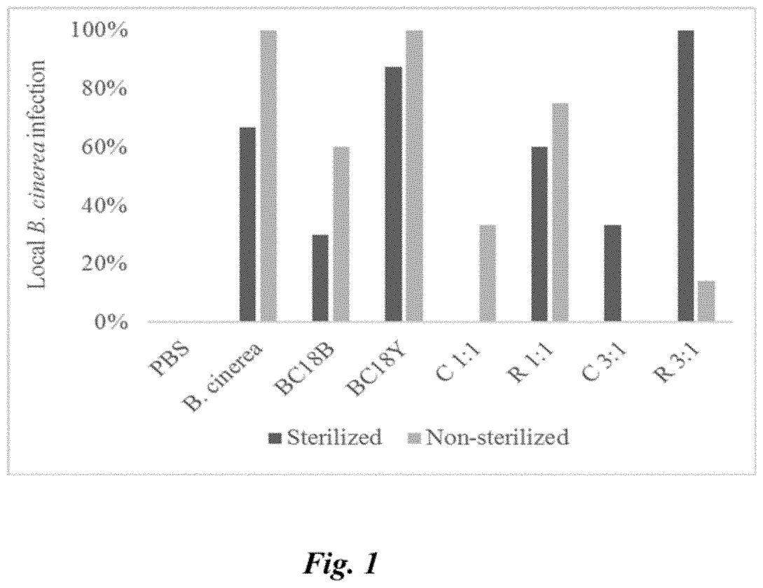

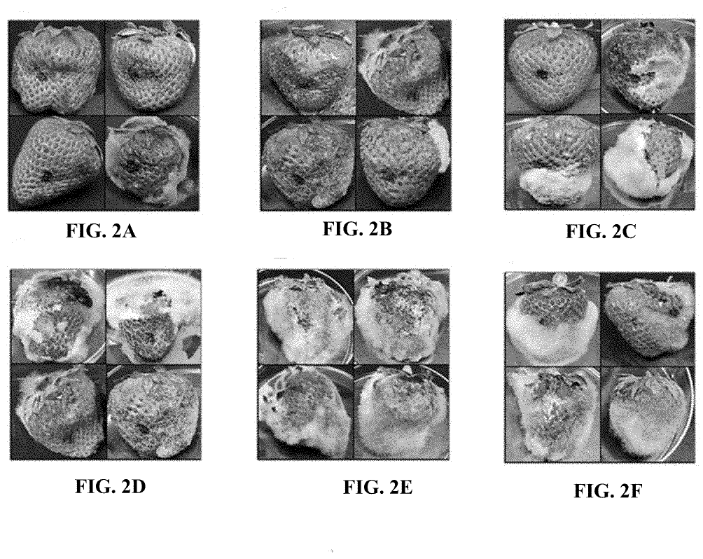

The microorganism consortium BC18 (comprised of Gluconobacter cerinus and Hanseniaspora uvarum) was tested for the ability to prevent Botrytis cinerea growth on post-harvest strawberry fruits. Microorganism components of BC18 were cultured in isolation, co-cultured together, or recombined after being cultured in isolation. Co-cultured BC18 resulted in decreased fungal disease incidence on whole strawberry fruits compared to BC18 microorganism components cultured as isolates or recombined into a consortium (FIG. 1 and FIGS. 2A-F).

### Experimental Setup

### Microorganism Growth Conditions

BC18 microorganism components were grown in 250 ml culture flasks with 50 ml potato dextrose broth for 72 hours at 28° C. with shaking at 150 rpm. After 72 hours, 30 ml of such shake flask broths were centrifuged at 3500 rpm for 10 minutes at 22° C. Cells were re-suspended in phosphate buffered saline (PBS; 100 mM phosphate buffer pH 7.0) to a concentration of 1×10⁸ cells/ml as counted on a hemocytometer with an Olympus Bx microscope. BC18 microorganism components used in this experiment consisted of: Gluconobacter cerinus cultured individually, Hanseniaspora uvarum cultured individually, and two co-cultures of G. cerinus and H. uvarum. The product ratio of G. cerinus and H. uvarum in each co-culture, at the end of fermentation was about 1:1 and 3:1, respectively, as counted by hemacytometer. G. cerinus cultured individually and H. uvarum cultured individually were combined after re-suspension in PBS to 1×10⁸ cells/mL in a 3:1 and 1:1 ratio (G. cerinus:H. uvarum).

B. cinerea was cultured on strawberry agar (comprising 500 g blended strawberry fruits, 500 g water, and 20 g agar) in 100 mm×15 mm petri plates for eight days at 25° C. Spores were collected by adding 15 mL of PBS to two such plates and scraping the plate with a sterile disposable L-shaped spreader. The resulting spore suspension was decanted into a 50 ml centrifuge tube through a 40 μm cell strainer. The spore suspension was centrifuged at 3500 rpm and 22° C. for ten minutes and re-suspended in sterile PBS to achieve a final spore concentration of 1×10⁶ spores per mL as counted on a hemocytometer.

### Strawberry Fruit Inoculation and Incubation

Bella Vista Organic strawberry fruits were purchased commercially at Sprouts Farmers Market (30 San Antonio Rd, Mountain View, Calif. 94040). Strawberry fruits were left either non-sterilized, in which case no modification was made to the strawberry fruit after purchase, or sterilized, in which case the entire surface of the strawberry fruit was wiped for 20-30 seconds with a disinfectant wipe (Good and Clean Inc.). Non-sterilized and sterilized strawberry fruits were each inoculated with one of the following treatments (N=10): sterile PBS, negative control; sterile PBS, positive control; G. cerinus, referred to as BC18B; H. uvarum, referred to as BC18Y; G. cerinus:H. uvarum co-cultured in a 1:1 ratio, referred to as C1:1; G. cerinus:H. uvarum co-cultured in a 3:1 ratio, referred to as C3:1; G. cerinus:H. uvarum combined in a 3:1 ratio, referred to as R3:1; G. cerinus:H. uvarum combined in a 1:1 ratio, referred to as a R1:1 ratio.

Inoculation was accomplished by creating an inoculation mark with a sharpie marker two-thirds down the length of the strawberry fruit. A 10 μl pipettor was used to insert 10 μl of microorganism candidate suspension or sterile PBS within 5 mm to the right of the inoculation mark, with the pipet tip inserted no more than half its length into the strawberry fruit. This allowed for inoculation of both the interior of the strawberry fruit and the exterior of the strawberry fruit where residual microorganism suspension or sterile PBS rested after inoculation.

Strawberry fruits were contained in one side of a sterile 100 mm×15 mm petri plate wrapped in heavy duty tin foil to prevent contamination between strawberry fruits. Inoculated strawberry fruits were incubated for 24 hours at 25° C. in the dark to allow microorganism colonization of the strawberry fruit. After 24 hours, the B. cinerea spore suspension was inoculated into the strawberry fruits as described above in the same place as the microorganism suspension or sterile PBS had been previously inoculated. The PBS negative controls received no B. cinerea inoculation.

### Experimental Analysis

Images of strawberry fruits were taken with an iPhone 7 at 3 and 6 days post B. cinerea inoculation (T3 and T6, respectively). At T3 none of the positive controls (receiving only sterile PBS and B. cinerea inoculation) showed signs of B. cinerea growth. Multiple strawberry fruits, however, were covered with other naturally occurring fungal pathogens such that the inoculation site was covered before B. cinerea had a chance to grow. These strawberries were removed from the analysis (Table 3). At T6 strawberry fruits were assessed for the presence or absence of B. cinerea growth at the inoculation site. If the presence or absence of B. cinerea could not be determined, i.e. due to an obscured inoculation site, then that strawberry fruit was excluded from analysis (Table 3). The number of strawberry fruits in each treatment with evidence of B. cinerea growth was divided by the total number of strawberry fruits remaining, per treatment, to calculate the percentage of local B. cinerea fungal disease incidence (LBDI).

TABLE 3
Development of LBDI in strawberry fruits after various
treatments prior to infection by B. cinerea

| | | SF | SF | |
|---|---|---|---|---|
| | | excluded | excluded | B. cinerea |
| Treatment | SF^{a} condition | at T3^{b} | at T6^{c} | incidence^{d} |
| PBS control | sterilized | 8 | 2 | N/A |
| B. cinerea control | sterilized | 7 | 0 | 2 |
| BC18B | sterilized | 0 | 0 | 3 |
| BC18Y | sterilized | 2 | 0 | 7 |
| C 1:1 | sterilized | 2 | 4 | 0 |
| R 1:1 | sterilized | 2 | 3 | 3 |
| C 3:1 | sterilized | 0 | 1 | 3 |
| R 3:1 | sterilized | 4 | 2 | 4 |
| PBS control | non-sterilized | 6 | 4 | N/A |
| B. cinerea control | non-sterilized | 2 | 1 | 7 |
| BC18B | non-sterilized | 3 | 2 | 3 |
| BC18Y | non-sterilized | 3 | 3 | 4 |
| C 1:1 | non-sterilized | 0 | 4 | 2 |
| R 1:1 | non-sterilized | 1 | 1 | 6 |
| C 3:1 | non-sterilized | 0 | 3 | 0 |
| R 3:1 | non-sterilized | 2 | 1 | 1 |

^{a}Strawberry Fruit
^{b}This column shows the number of strawberry fruits eliminated from each treatment at T3 due to over-growth of naturally occurring fungal diseases which obscured the B. cinerea inoculation site.
^{c}This column shows the number of strawberry fruits at T6 for which the LBDI could not be determined. These strawberry fruits were not used in % LBDI calculation.
^{d}Number of strawberry fruits showing evidence of B. cinerea growth at the inoculation site.

For both the sterilized and non-sterilized strawberry fruits, the co-cultured BC18 out-performed the each of the two individual BC18 microorganism components (BC18B and BC18Y) as individually cultured isolates, and the combination of the two individually cultured isolates. While BC18B did show a small reduction in LBDI compared to the positive control, BC18Y did not show reduced LBDI on either sterilized or non-sterilized strawberry fruits. For non-sterilized strawberry fruits, C3:1 had 0% LBDI and its counter-part, R3:1 had a 14% LBDI. C1:1 had a 33% LBDI while the R1:1 treatment had a 75% LBDI. Likewise, on sterilized strawberry fruits, C3:1 had a 67% less LBDI than R3:1 and C1:1 had 60% less LBDI than R1:1 (FIG. 1 and FIGS. 2A-F). FIGS. 2A-2F show representative images from 6 days post B. cinerea inoculation of strawberry fruits inoculated with co-cultured BC18 compared to the recombined BC18 counterpart. Specifically, FIG. 2A shows C3:1, FIG. 2B shows C1:1, FIG. 2C shows R3:1, FIG. 2D shows R1:1, FIG. 2E shows BC18Y, FIG. 2F shows a B. cinerea only control.

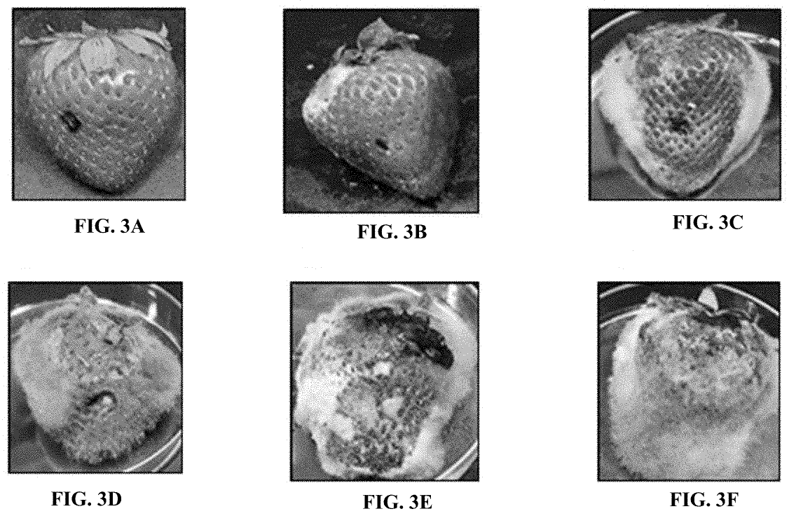

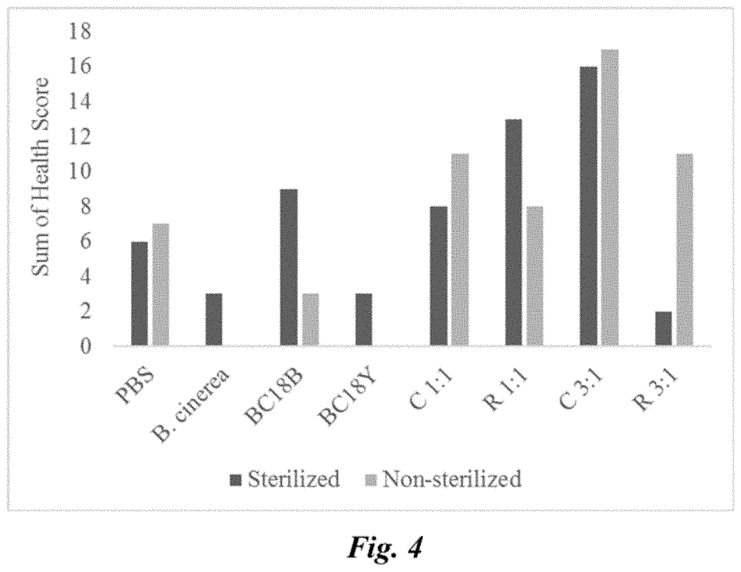

It should be noted that, while each BC18 co-culture had increased efficacy over the combined counter-part, C3:1 had increased efficacy on non-sterile strawberry fruits and C1:1 had the best efficacy on sterile strawberry fruits. Without being limited by theory, this may be related to the disruption of the native strawberry fruit surface microbiome during sterilization and indicates that the ratio of the BC18 co-culture influences its activity on strawberry fruit surfaces. The presence of naturally occurring fungal pathogens granted an opportunity to observe how well a localized inoculation of BC18 consortium protected the entire strawberry fruit against other fungal disease, most prominently Rhizopus. These observations were quantified by assigning a health score to each strawberry based on the fungal disease incidence (FDI) and the FDI proximity to the inoculation site (FIG. 3A-F). FIG. 3A shows 4-point strawberry fruit which has no fungal disease evident. FIG. 3B shows a 3-point strawberry fruit which has fungal disease present on strawberry fruit, but not near the inoculation site. FIG. 3C shows a 2-point strawberry which has fungal disease is within an estimated 5 mm of inoculation site. FIG. 3D shows a 1-point strawberry which has fungal disease that is at the edge of the inoculation site. FIG. 3E shows a 1-point strawberry which has fungal disease not present at the edge of the inoculation site, but the inoculation site is unhealthy. FIG. 3F shows a 0-point strawberry which has fungal disease covering the strawberry fruit irrespective of inoculation site. FIG. 4 shows the summation of health scores per treatment for each strawberry fruit. Strawberry fruits that were eliminated from analysis at T3 were assumed to have a health score of 0. Strawberry fruits inoculated with C3:1 had the highest health scores (FIG. 4), far out-performing strawberry fruits inoculated with R3:1. From the results, both the co-culture condition and the ultimate ratio of G. cerinus to H. uvarum in the co-culture may influence the efficacy of BC18 against FDI on strawberry fruits.

### Example 2: Fermentation of Co-Culture of Hanseniaspora uvarum and Gluconobacter cerinus Resulted in Higher Viable Biomass than Either Microorganism Fermented Individually

Three co-culture fermentation experiments (conditions: co-culture control, co-culture with feed off, co-culture with feed off and temp spike) and one fermentation experiment of Hanseniaspora uvarum alone (condition: H. uvarum alone), were performed in 2-liter (2-L) benchtop DASGIP fermentors. A medium consisting of yeast extract (5-10 g/kg), magnesium sulphate heptahydrate (1-3 g/kg), potassium phosphate monobasic (0.5-2 g/kg), ammonium sulphate (0.5-1.5 g/kg), trace elements solution similar to Modified Trace Metals Solution from Teknova and vitamins solutions (2 mL/kg each) along with antifoam (1 g/kg) was used for all fermentations. Vitamin solution was made consisting of Pantothenic acid (2-4 g/L), thiamine HCl (1-6 g/L), riboflavin (0.25-2.25 g/L), pyridoxine HCl (0.25-2.25 g/L) and biotin (0.25-2.25 g/L) and was foil-wrapped and store in the refrigerator at 4° C. Calcium chloride dihydrate (2-4 g/L) and glucose (50 g/L) was added as post-sterile. pH and temperature for the yeast fermentors was 4.8 and 29° C. respectively; whereas co-culture fermentations ran at pH 5.2 and temperature 30° C. pH control was done using aqueous ammonia. The feed consisting of 50% w/w glucose solution was fed starting 20 hrs until end of the run at 68 hrs at 7.4 mL/hr rate. Three co-culture fermentations were run in identical manner throughout the run except two fermentations out of three were given different end of fermentation treatment. For one fermentation (condition: co-culture with feed off), at 67 hrs, feed was shut off. The last co-culture fermentation (condition: co-culture with feed off and temp spike) had feed shut off and temperature was increased to 32° C. at 67 hrs.

One fermentation experiment of Gluconobacter cerinus alone (condition: G. cerinus alone), was done in 15 L SIP/CIP fermentor. The fermentation media consisted of—yeast extract (5-10 g/kg), soymeal (5-10 g/kg), magnesium sulphate heptahydrate (1-3 g/kg), potassium phosphate monobasic (0.5-2 g/kg), ammonium sulphate (0.5-1.5 g/kg), trace elements solution similar to Modified Trace Metals Solution from Teknova (2 mL/kg) along with antifoam (1 g/kg). Calcium chloride dihydrate (2-4 g/L) and glucose (50 g/L) was added as post-sterile. pH was controlled at 5.5 and temperature was 30° C. pH control was done using aqueous ammonia. The feed consisting of 60% w/w glucose solution was fed starting 30 hrs until end of the run (72 hrs) at 0.95 g/min rate.

G. cerinus alone fermentation experienced a lot of foaming, requiring significant amounts of antifoam addition during the fermentation process; whereas co-culture fermentations did not experience any foaming, thereby making it more scalable process.

Viability of each end of fermentation sample was measured by serial dilution plating on potato dextrose agar. CFU (colony forming unit) plating was done by serial diluting sub-samples of each sample in a 96-well plate using potato dextrose broth and plating 20 μl of a dilution range that is likely to generate countable colonies at certain timepoints on potato dextrose agar. Plates were incubated for 2 days at room temperature. Colonies were counted manually and multiplied by the dilution factor 50 to determine CFU/mL (colony forming unit/milliliter). Only the highest countable dilution is used for final calculation of CFU/mL.

Co-culturing the two microorganisms results in two log increase in viable biomass at the end of fermentation process. Table 5 demonstrates the CFU/mL (colony forming unit/milliliter) at the end of fermentation for the various conditions and microbes. As shown in Table 5, co-culturing resulted in at least a log increase compared to the total viable cell counts obtained from H. uvarum and G. cerinus alone.

TABLE 5
Viable cell counts at the end of fermentation

| | | | | | Co-culture with |
|---|---|---|---|---|---|
| | H. uvarum | G. cerinus | Co-culture | Co-culture | feed off and temp |
| Condition | alone | alone | control | with feed off | spike |
| CFU/mL at | 8.50 × 10⁹ | 1.80 × 10⁹ | 2.13 × 10¹¹ | 1.90 × 10¹¹ | 1.25 × 10¹¹ |
| End of | | | | | |
| fermentation | | | | | |

### Example 3. Co-Culture of Hanseniaspora uvarum and Gluconobacter cerinus Demonstrated Improvement in Stability Compared to Either Microorganism Alone

End of fermentation samples from Example 2 were stored in the refrigerator at 4° C. Viability was measured using the same serial dilution plating method described in Example 2, at 33 days and 50 days for sample containing bacteria alone and 31 days and 46 days for yeast and co-culture. At 31 days, dilutions 10⁻⁶, 10⁻⁷ and 10⁻⁸ were plated. At 33 days, dilutions 10⁻⁴, 10⁻⁵ and 10⁻⁶ were plated. At 46 days, dilutions 10⁻⁴, 10⁻⁵ and 10⁻⁶ were plated for yeast alone sample and dilutions 10⁻⁷ and 10⁻⁸ were plated for co-culture. At 50 days, dilutions 10⁻⁷ and 10⁻⁸ were plated.

The H. uvarum alone fermentation sample stored at 4° C. for over a month didn't show any growth on dilution plates whereas both H. uvarum and G. cerinus when fermented individually did not show any growth on dilution plates after samples had been stored for 50 days. Co-culture showed no more than 1.5 log drop in viability counts during extended storage at 4° C. conditions for up to 50 days.

All co-culture samples regardless of differences in end of fermentation treatments have superior stability compared to fermentation samples of individual microorganisms. Table 6 below shows the viable cell counts from each case at each timepoint.

TABLE 6
Viable cell counts of microbes over the course of time

| | CFU/mL at days | | |
|---|---|---|---|
| Condition | 0 | 31-33 | 46-50 |
| H. uvarum alone | 8.50 × 10⁹ | <10³ | <10³ |
| G. cerinus alone | 1.80 × 10⁹ | 4.73 × 10⁹ | <10⁶ |
| Co-culture control | 2.13 × 10¹¹ | 1.09 × 10¹² | 4.05 × 10¹⁰ |
| Co-culture with feed off | 1.90 × 10¹¹ | 1.02 × 10¹⁰ | 2.15 × 10¹⁰ |
| Co-culture with feed off | 1.25 × 10¹¹ | 7.90 × 10⁹ | 6.50 × 10⁹ |
| and temp spike | | | |

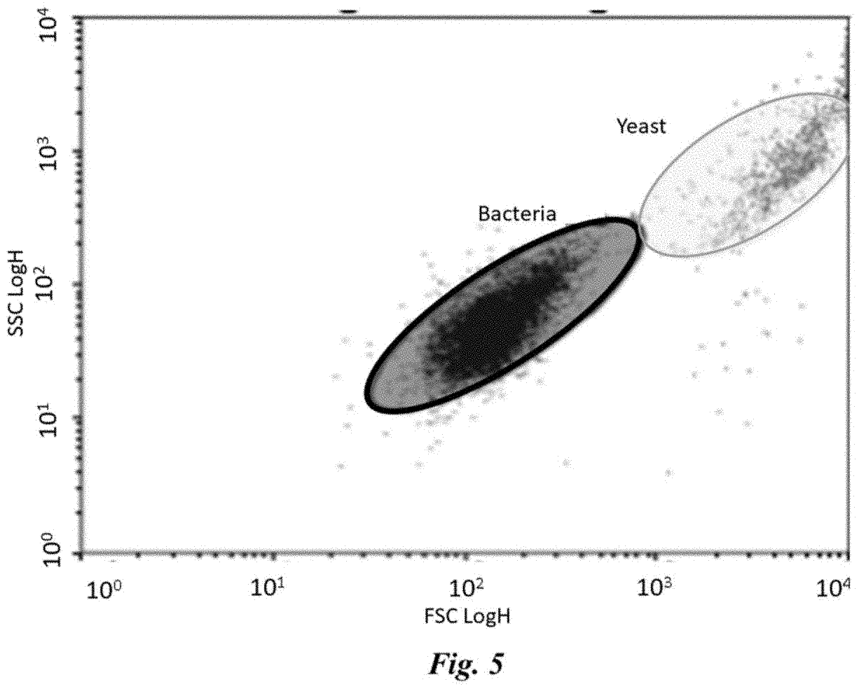

H. uvarum to G. cerinus ratios for all co-culture fermentation samples were measured at the end of fermentation and after 46 days storage in spent fermentation broth at 4° C. End of fermentation ratios were calculated by flow cytometry, using a Stratedigm S100. Samples were centrifuged at 3500 rpm for 10 minutes at 22° C. Pelleted solids were then re-suspended in an equivalent volume of sterile PBS. Suspensions were passed by gravity through a 20 μm mesh filter and 100 μl of the filtrate added to 1 mL of PBS. As H. uvarum is both larger and more internally complex than G. cerinus a clear separation of each cell population was seen using forward and side scatter parameters (FIG. 5). The H. uvarum to G. cerinus ratios after 46 days in storage were calculated by microscopy combined with manual counts. Wet mount slides were imaged at 40× magnification in phase contrast on a Leica DM5500 B light microscope. The number of H. uvarum and G. cerinus in three such images per sample were manually counted to determine the ratio of microbial components in each sample. Table 7 shows the ratios of the microorganisms in the co-culture after storage at 4° C. It is noteworthy that in all cases G. cerinus is present in much higher concentrations than the H. uvarum. However, even though the co-culture is dominated by G. cerinus, co-culture viability is superior compared to viability of either organism cultured individually.

TABLE 7
Ratios of microorganisms within co-culture
samples after storage at 4° C.

Ratio of G. cerinus to H. uvarum,
days after fermentation

| Condition | 0 days | 46 days |
|---|---|---|
| Co-culture control | 49:1 | 49:1 |
| Co-culture with feed off | 3:1 | 99:1 |
| Co-culture with feed off | 99:1 | 33:1 |
| and temp spike | | |

### Example 4. Co-Cultured BC18 on Strawberry in Fields and Post-Harvest

Co-cultured BC18 is assessed for efficacy against Botrytis cinerea in strawberry fields. Co-cultured BC18 is applied to plots at a dosage less than 10⁸ cfu/acre with less than 4 application per month. Additionally, to test the efficacy of co-cultured BC18 after storage, a set of co-cultured BC18 is stored at 25° C. for four weeks prior to application, with different dosages to test for a loss of activity due to storage. Both fresh (unaged) co-cultured BC18 and BC18 that has been stored for four weeks are applied to plot of strawberries. Multiple replicates of each experimental condition are performed. Controls plots are left untreated or treated with another compound (as a biological benchmark). Additionally, in a separate plot co-cultured BC18 are applied along with a standard schedule of fertilizer, fungicides and/or insecticides commonly used in Integrated Pest Managements to determine compatibility and to observe any adverse effects on any of the compositions used on the strawberries. Example of other fungicides that may be applied include, but are not limited to, fluopyram, aluminum tris (O-ethyl phosphonate), azoxystrobin, boscalid, captan, fenhexamid, copper hydroxide, copper oxychloride, copper sulfate, cuprous oxide, cyprodinil, fludioxonil, fenhexamid, fluoxastrobin, iprodione, mefenoxam, metalaxyl, myclobutanil, phosphite (phosphorous acid salts), propiconazole, pyraclostrobin, pyrimethanil, quinoxyfen, sulfur, thiophanate-methy, trifloxystrobin, or triflumizole. Examples of insecticides include, but are not limited to, acetamiprid, benifenthrin, fenpropathrin, endosulfan, novaluron, or carbaryl.

Strawberries are observed in the field and post-harvest to determine the inhibition of Botrytis cinerea. Strawberries in the field and post-harvest are photographed and scored to determine the health of the strawberries. The inhibition is compared to a competitive benchmark to determine improved efficacy of co-cultured BC18 over a benchmark.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A biocontrol composition comprising at least two microbes, wherein the at least two microbes comprise:
(a) a Gluconobacter cerinus, and
(b) a Hanseniaspora uvarum;
wherein the at least two microbes are co-cultured, wherein the at least two microbes are co-cultured at a product ratio.

2. The biocontrol composition of claim 1, wherein the product ratio of the Gluconobacter cerinus and the Hanseniaspora uvarum is between about 1:100 and 100:1.

3. The biocontrol composition of claim 1, wherein the product ratio of the Gluconobacter cerinus and the Hanseniaspora uvarum is between about 1:10 and 10:1.

4. (canceled)

5. The biocontrol composition of claim 1, wherein the product ratio of the Gluconobacter cerinus and the Hanseniaspora uvarum is between about 1:3 and 3:1.

6. The biocontrol composition of claim 1, wherein the product ratio of the Gluconobacter cerinus and the Hanseniaspora uvarum is between about 1:2 and 2:1.

7. The biocontrol composition of claim 1, wherein the biocontrol composition is capable of inhibiting a fungal disease incidence by 10% or more compared to a reference composition comprising any composition selected from the group consisting of: (i) one or more of the at least two microbes cultured individually or (ii) the at least two microbes cultured separately and combined at a viable cell count and product ratio that is about the same as that of the biocontrol composition.

8. A biocontrol composition of claim 1, wherein a viable cell count at the end of fermentation of the co-cultured at least two microbes, grown using a given fermentation medium, feed composition and process, is more than five times than a sum of the viable cell counts of the at least two microbes at the end of an equivalent fermentation process.

9. (canceled)

10. A biocontrol composition of claim 1, wherein a viable cell count at the end of fermentation of the co-cultured at least two microbes, grown using a given fermentation medium, feed composition and process, is more than two times than a sum of the viable cell counts of the at least two microbes at the end of an equivalent fermentation process.

11. A biocontrol composition of claim 1, wherein a viable cell count of the at least two microbes after being subjected to a storage condition, is higher than a sum of viable cell counts of the at least two microbes grown alone in an equivalent fermentation process and under the storage condition.

12. The biocontrol composition of claim 11, wherein the storage condition comprises storage at a temperature between 4° C. and 25° C.

13. The biocontrol composition of claim 11, wherein the storage condition comprises a storage time of at least 7 days.

14. A method of generating the biocontrol composition of claim 1, comprising:
(a) introducing a first microbe of the at least two microbes to a first culturing medium;
(b) introducing a second microbe of the at least two microbes to a second culturing medium, wherein the second culturing medium comprises: the first culturing medium or a derivative thereof, the first microbe, or a combination thereof, wherein the second microbe is different from the first microbe; and
(c) subjecting the first microbe and second microbe to conditions to allow cell proliferation, thereby generating the biocontrol composition.

15. The method of claim 14, wherein the second culturing medium is the first culturing medium after conditioning by the first microbe.

16. The method of claim 14, wherein the first microbe is Gluconobacter cerinus and the second microbe is Hanseniaspora uvarum.

17. The method of claim 14, wherein the first microbe is Hanseniaspora uvarum and the second microbe is Gluconobacter cerinus.

18. A method of reducing or preventing growth of a pathogen on a plant, a seed, a flower or produce thereof comprising: (i) applying the biocontrol composition of claim 1 to a plant, a seed, a flower or produce thereof, or (ii) applying the biocontrol composition of claim 1 to a packaging material comprising the plant, seed, flower or produce thereof.

19. The method of claim 18, wherein the plant, seed, flower, or produce thereof is selected from the group consisting of alfalfa, almond, apricot, apple, artichoke, banana, barley, beet, blackberry, blueberry, broccoli, Brussels sprout, cabbage, cannabis, canola, capsicum, carrot, celery, chard, cherry, citrus, corn, cotton, cucurbit, date, fig, flax, garlic, grape, herb, spice, kale, lettuce, mint, oil palm, olive, onion, pea, pear, peach, peanut, papaya, parsnip, pecan, persimmon, plum, pomegranate, potato, quince, radish, raspberry, rose, rice, sloe, sorghum, soybean, spinach, strawberry, sweet potato, tobacco, tomato, turnip greens, walnut, and wheat.

20. The method of claim 19, wherein the plant, seed, flower, or produce thereof comprises a strawberry.

21-25. (canceled)

26. The method of claim 18, wherein the pathogen is selected from the group consisting of: Albugo candida, Albugo occidentalis, Alternaria alternata, Alternaria cucumerina, Alternaria dauci, Alternaria solani Alternaria tenuis, Alternaria tenuissima, Alternaria tomatophila, Aphanomyces euteiches, Aphanomyces raphani, Armillaria mellea Aspergillus flavus, Aspergillus parasiticus, Botrydia theobromae, Botrytis cinerea, Botrytinia fuckeliana, Bremia lactuca, Cercospora beticola, Cercosporella rubi, Cladosporium herbarum, Colletotrichum acutatum, Colletotrichum gloeosporioides, Colletotrichum lindemuthianum, Colletotrichum musae, Colletotrichum spaethanium, Cordana musae, Corynespora cassiicola, Daktulosphaira vitifoliae, Didymella bryoniae, Elsinoe ampelina, Elsinoe mangiferae, Elsinoe veneta, Erysiphe cichoracearum, Erysiphe necator, Eutypa lata, Fusarium germinareum, Fusarium oxysporum, Fusarium solani, Fusarium virguliforme, Gaeumannomyces graminis, Ganoderma boninense, Geotrichum candidum, Guignardia bidwellii, Gymnoconia peckiana, Helminthosporium solani, Leptosphaeria coniothyrium, Leptosphaeria maculans, Leveillula taurica, Macrophomina phaseolina, Microsphaera alni, Monilinia fructicola, Monilinia vaccinii-corymbosi, Mycosphaerella angulate, Mycosphaerella brassicicola, Mycosphaerella fragariae, Mycosphaerella fijiensis, Oidopsis taurica, Passalora fulva, Penicillium expansum, Peronospora sparse, Peronospora farinosa, Pestalotiopsis clavispora, Phoma exigua, Phomopsis obscurans, Phomopsis vaccinia, Phomopsis viticola, Phytophthora capsica, Phytophthora erythroseptica, Phytophthora infestans, Phytophthora parasitica, Phytophthora ramorum, Plasmopara viticola, Plasmodiophora brassicae, Podosphaera macularis, Polyscytalum pustulans, Pseudocercospora vitis, Puccinia allii, Puccinia sorghi, Pucciniastrum vaccinia, Pythium aphanidermatum, Pythium debaryanum, Pythium sulcatum, Pythium ultimum, Ralstonia solanacearum, Ramularia tulasneii, Rhizoctonia solani, Rhizopus arrhizus, Rhizopus stoloniferz, Sclerotinia minor, Sclerotinia homeocarpa, Sclerotium cepivorum, Sclerotium rolfsii, Sclerotinia minor, Sclerotinia sclerotiorum, Septoria apiicola, Septoria lactucae, Septoria lycopersici, Septoria petroelini, Sphaceloma perseae, Sphaerotheca macularis, Spongospora subterrannea, Stemphylium vesicarium, Synchytrium endobioticum, Thielaviopsis basicola, Uncinula necator, Uromyces appendiculatus, Uromyces betae, Verticillium albo-atrum, Verticillium dahliae, Verticillium theobromae, and any combination thereof.

27. The method of claim 18, wherein the pathogen is Botrytis cinerea.
